(19) United States
(12) Patent Application Publication
(10) Pub. No.: US 2026/0137620 A1
(43) Pub. Date: May 21, 2026

(54) PHARMACEUTICAL EMULSION PROCESS

(71) Applicant: Fresenius Kabi USA, LLC, Lake Zurich, IL (US)

(72) Inventor: Chaoju XIAO, Lake Zurich, IL (US)

(73) Assignee: Fresenius Kabi USA, LLC, Lake Zurich, IL (US)

(21) Appl. No.: 19/119,589

(22) PCT Filed: Oct. 16, 2023
(86) PCT No.: PCT/US2023/035232

§ 371 Date: Apr. 9, 2025

Related U.S. Application Data
(60) Provisional application No. 63/379,788, filed on Oct. 17, 2022.

(51) Int. Cl. A61K 9/107; A61K 31/5377; A61K 47/10; A61K 47/12; A61K 47/24; A61K 47/26; A61K 47/44

(52) U.S. Cl. CPC A61K 9/107; A61K 31/5377; A61K 47/10; A61K 47/12; A61K 47/24; A61K 47/26; A61K 47/44

(57) ABSTRACT

Provided is a process for preparing an emulsion having an oil phase containing an emulsifier, an oil, a drug, and a co-emulsifier, and an aqueous phase containing water, a tonicity agent, and a pH modifier, without removing excess co-emulsifier after emulsification. The process includes emulsifying an oil phase containing an oil, aprepitant, an egg lecithin, and ethanol, and an aqueous phase containing water, sucrose, and oleic acid or salt thereof, without removing ethanol after emulsification. The drug is added to the oil phase as a dry mixture containing the drug and a co-emulsifier (e g., ethanol).

### BACKGROUND OF THE INVENTION

Pharmaceutical emulsions include dispersions or suspensions of globules or droplets of a liquid (e.g., an oil phase) containing an active pharmaceutical ingredient (e.g., active agent, medicament, or drug product), distributed throughout a fluid medium (e.g., an aqueous phase) in which the globules or droplets are immiscible. Pharmaceutical emulsions are often used for delivering poorly water-soluble active pharmaceutical ingredients in a fluid medium. Such emulsions may be useful for, e.g., oral, topical, intramuscular, subcutaneous, or intravenous administration, and may be formulated as, e.g., an oil in water or water-in-oil emulsion, or as a more complex emulsion system, e.g., a double or multiple emulsion such as, e.g., a water-in-oil-in-water or oil-in-water-in-oil emulsion. Pharmaceutical emulsions also may improve drug solubilization and/or distribution in aqueous dilution media including sterile aqueous media used for administration by injection, e.g., sterile saline, sterile water for injection, sterile aqueous dextrose solutions, etc. Pharmaceutical emulsions additionally may provide formulations that are storage stable and provide desirable pharmacokinetic properties for delivering the active agent, e.g., desirable blood levels of the active agent, bioavailability, etc.

In formulating pharmaceutical emulsions, it is important to produce the formulation as efficiently as possible while maintaining stability of the active pharmaceutical ingredient throughout the production process. Formulating emulsions of drugs that are both poorly soluble in water and difficult to dissolve a pharmaceutically acceptable oil phase may be challenging, particularly for poorly water soluble drugs that are sensitive to stresses that may occur during the production process, e.g., high temperatures, shear forces, the use of pH adjusting agents, etc., and difficult to dissolve in an oil phase without the aid of a co-emulsifier (used in addition to an emulsifier that promotes formation and stabilization of the emulsion).

Aprepitant is one example of an active pharmaceutical agent that is delivered as an emulsion (oil-in-water), which is challenging to formulate, in part, due to difficulty associated with solubilizing the drug in an oil phase, conventionally requiring the use of excess of co-emulsifier (e.g., ethanol in excess of pharmaceutically acceptable levels) to dissolve to drug in the oil phase, and then removal of excess co-emulsifier (e.g., ethanol) following emulsification in order to achieve pharmaceutically acceptable levels of the co-emulsifier in the final product. For example, ethanol levels in injectable pharmaceuticals must be minimized to avoid adverse effects in patients, including, for example, pain and hemolysis. See, e.g., Serdons et al., The Presence of Ethanol in Radiopharmaceutical Injections, J. Nuclear Med. 2008, 49 (12) 2071. However, conventional methods of formulating injectable oil-in-water emulsions of aprepitant require using ethanol in excess of pharmaceutical levels to solubilize the aprepitant in the oil phase, and then removing excess ethanol following emulsification in order to achieve acceptable levels. For example, U.S. Patent Application Publication No. US20130317016A1 (Higorani et al.) describes a method of solubilizing aprepitant using 60% volume % ethanol, with a final sterile liquid formulation comprising about 30% volume % ethanol. Similarly. U.S. Pat. No. 9,561,229 (Ottoboni et al.) describes the preparation of sterile oil-in-water emulsions containing aprepitant (presently marketed as CinvantiR) that contains an emulsifier, co-emulsifier, oil, tonicity agent, pH-modifying agent, buffer, and water. The emulsion described by Ottoboni et al, comprises an oil phase consisting of aprepitant, egg lecithin, and ethanol added to soy bean oil, and an aqueous phase comprising sucrose, sodium oleate, and water. Ottoboni et al. describes aprepitant as a molecule with poor solubility and poor permeability characteristics, and provides examples of preparing aprepitant emulsions with an excess of ethanol as a co-emulsifier, which is later removed to various levels by evaporation methods. The additional step of having to remove excess ethanol following emulsification is time consuming and costly, and exposes the active agent to additional stresses during the formulation process.

Accordingly, there is a need for improved methods for preparing pharmaceutical emulsions, particularly oil-in-water emulsions that contain a poorly water soluble active pharmaceutical ingredient which requires a co-emulsifier to produce the emulsion, e.g., Cinvanti®. The present invention provides such a method.

### BRIEF SUMMARY OF THE INVENTION

The present invention provides a process for preparing an emulsion, which process includes emulsifying (i) an oil phase containing at least one oil, at least one active pharmaceutical ingredient (API), at least one solid emulsifier, and at least one co-emulsifier, and (ii) an aqueous phase containing water, at least one tonicity agent, and at least pH modifier, wherein the co-emulsifier is needed to dissolve the API effectively in the oil phase. i.e., wherein dissolving the API in the oil phase effectively requires the aid of co-emulsifier. The process of the invention includes combining a dry mixture containing at least one active pharmaceutical ingredient (API) and at least a portion of a solid emulsifier (e.g., combining the API as a pre-mixed powder containing the API and a solid emulsifier), with an oil, co-emulsifier, and, optionally, an additional quantity of the solid emulsifier, to produce the oil phase, combining the oil phase with an aqueous phase containing the water, tonicity agent, and pH modifier, and homogenizing the oil and aqueous phases to produce a pharmaceutically acceptable emulsion, wherein the co-emulsifier is present at a pharmaceutically acceptable level following emulsification thereby avoiding the need to remove excess co-emulsifier after emulsification.

In one embodiment, the invention provides a process for preparing an emulsion, which process includes emulsifying (i) an oil phase containing an oil, an active pharmaceutical ingredient (API) which includes aprepitant, a solid emulsifier which includes an egg yolk lecithin, and a co-emulsifier which includes ethanol, and (ii) an aqueous phase containing water, a tonicity agent which includes sucrose, and a pH modifier which includes oleic acid or a salt thereof, wherein the ethanol is present at a pharmaceutically acceptable level following emulsification without removing (e.g., evaporating) ethanol from the emulsion. The process includes combining a dry mixture containing aprepitant and at least a portion of an egg yolk lecithin (e.g., adding the aprepitant as a pre-mixed powder containing the aprepitant and egg yolk lecithin), with an oil, ethanol, and, optionally, an additional quantity of egg yolk lecithin, to produce the oil phase, combining the oil phase with an aqueous phase containing water, sucrose, and oleic acid or a salt thereof, and homogenizing the oil and aqueous phases to produce a pharmaceutically acceptable emulsion containing a pharmaceutically acceptable level of ethanol following emulsification without a step of removing (e.g., evaporating) ethanol from the emulsion.

### DETAILED DESCRIPTION OF THE INVENTION

The present invention provides a process for preparing an emulsion, which process includes emulsifying (i) an oil phase containing at least one oil, at least one active pharmaceutical ingredient (API), at least one solid emulsifier, and at least one co-emulsifier, and (ii) an aqueous phase containing water, at least one tonicity agent, and at least pH modifier, wherein the co-emulsifier is needed to dissolve the API effectively in the oil phase, i.e., wherein dissolving the API in the oil phase effectively requires the aid of co-emulsifier. The process of the invention includes combining a dry mixture containing at least one active pharmaceutical ingredient (API) and at least a portion of a solid emulsifier (e.g., combining the API as a pre-mixed powder containing the API and a solid emulsifier), with an oil, co-emulsifier, and, optionally, an additional quantity of the solid emulsifier, to produce the oil phase, combining the oil phase with an aqueous phase containing the water, tonicity agent, and pH modifier, and homogenizing the oil and aqueous phases to produce a pharmaceutically acceptable emulsion, wherein the co-emulsifier is present at a pharmaceutically acceptable level following emulsification without removing any co-emulsifier from the emulsion.

The process of the invention is predicated, at least in part, on the surprising and unexpected discovery that adding the API as a dry mixture containing a solid emulsifier, e.g., as a pre-mixed powder containing the API and at least a portion of the solid emulsifier, reduces the amount co-emulsifier needed to dissolve the API effectively in the same oil system, i.e., as compared to adding the same amount of API by itself, e.g., as a powder not pre-mixed with a solid emulsifier, to the same oil system (with same emulsifiers, etc.) to produce the oil phase under the same conditions. The amount of co-emulsifier needed to dissolve the API effectively in the oil phase is reduced sufficiently to obviate the need to remove excess co-emulsifier, such that the inventive process produces the emulsion with pharmaceutically acceptable levels of co-emulsifier directly after emulsification of the oil and aqueous phases, without having to remove excess co-emulsifier following emulsification.

The process of the invention thus improves the efficiency of pharmaceutical emulsion processes that require using excess co-emulsifier to produce an oil phase of an emulsion, followed by removal of excess co-emulsifier from the emulsion following emulsification, e.g., to comply with regulatory requirements limit the amount of co-emulsifier in such emulsions. The method of the invention is particularly advantageous for processes of producing pharmaceutical emulsions that involve using an excess of a volatile co-emulsifier, e.g., ethanol, to dissolve an API in an oil phase, and then removing excess co-emulsifier following emulsification of the oil and aqueous phases, e.g., by evaporation, to achieve pharmaceutically acceptable levels of co-emulsifier in the emulsion. The method of the invention thus provides an improved process for producing pharmaceutical emulsions with an oil phase containing an oil, an API, a solid emulsifier, and a co-emulsifier, and an aqueous phase containing water, a tonicity agent, and a pH modifier, by reducing the amount of co-emulsifier (e.g., ethanol) needed to dissolve in the API effectively in the oil phase sufficiently to obviate the need to remove excess co-emulsifier (e.g., excess ethanol) following emulsification, e.g., to comply with regulatory requirements that limit the amount of co-emulsifier (e.g., ethanol) in such emulsions.

In one embodiment, the invention provides a process for producing a pharmaceutically acceptable emulsion, which process includes (a) mixing an active pharmaceutical ingredient (API) with a solid emulsifier to form a dry mixture, (b) mixing an oil with a co-emulsifier to form a first oil phase, (c) combining the dry mixture with the first oil phase to form a second oil phase, (d) combining water, a tonicity agent, and a pH modifier to form an aqueous phase, and (e) homogenizing the second oil phase with the aqueous phase to produce the emulsion without removing any co-emulsifier (e.g., without evaporating any volatile co-emulsifier if a volatile co-emulsifier is used).

The dry mixture produced in step (a) (mixing an active pharmaceutical ingredient (API) with a solid emulsifier to form a dry mixture) may be prepared by any method suitable for combining the API with the solid emulsifier, e.g., by dry mixing the API with the solid emulsifier. For example, the dry mixture may be produced by combining the API and a suitable quantity of solid emulsifier in a dry (e.g., solid) state, and mechanically agitating the API and solid emulsifier together as dry components, e.g., in a mixing bag or other suitable vessel, sufficiently to mix the components.

The first oil phase produced step (b) (mixing an oil with a co-emulsifier to form a first oil phase) may be prepared by methods that are known in the art. For example, the first oil phase may be produced by combining the oil and co-emulsifier and, optionally, further including a portion of solid emulsifier, in a suitable compounding tank, and mixing the components, e.g., at ambient temperature, until the components are dissolved.

The second oil phase produced in step (c) (combining the dry mixture with the first oil phase to form a second oil phase) may be prepared by methods that are known in the art. For example, the second oil phase may be produced by adding the dry mixture containing the API and solid emulsifier to the first oil phase while mixing in a suitable compounding tank, mixing the components, e.g., at ambient temperature, and optionally warming the mixture (e.g., at about 55-65° C.) while mixing until the dry mixture is dissolved. In some embodiments, the process for producing the second oil phase, e.g., as in step (c), further includes applying a vacuum to the second oil phase to evacuate the second oil phase, backfilling the evacuated second oil phase with an inert gas, e.g., nitrogen, and then heating the second oil phase in an atmosphere of the inert gas until the dry mixture completely dissolves therein. For example, the second oil phase may be produced by combining the dry mixture containing the API and solid emulsifier with the first oil phase in a suitable vessel, e.g., a compounding tank, applying a vacuum to the vessel to evacuate the vessel, e.g., sufficiently to remove at least a substantial portion of oxygen therefrom, backfilling with the evacuated vessel with an inert gas, e.g., nitrogen, and then, if needed, heating the vessel in a nitrogen atmosphere until complete dissolution of the dry mixture in the second oil phase.

The aqueous phase produced in step (d) (combining water, a tonicity agent, and a pH modifier to form an aqueous phase) may be prepared by methods that are known in the art. For example, the aqueous phase may be produced by adding appropriate amounts of tonicity agent and pH modifier to a suitable compounding tank containing an appropriate volume of water, e.g., water for injection, and mixing the components, e.g., at ambient temperature or above (e.g., at about 40-50° C.) until the tonicity agent and pH modifier are dissolved.

The emulsion produced in step (e) (homogenizing the second oil phase with the aqueous phase to produce the pharmaceutically acceptable emulsion) may be prepared by homogenizing the second oil phase and aqueous phase using methods that are known in the art. For example, the emulsification process may be carried out by transferring the second oil phase to the aqueous phase contained in a suitable compound tank while mixing at low speed (e.g., 200-500 rpm, 300-400 rpm, or 350 rpm), followed by homogenization of the oil and water phases at high speed (e.g., 1000-5000 rpm. 2000-3000 rpm, or 2500 rpm), and then microfluidization of the oil and water phases (e.g., at 20,000-40,000 psi, 25,000-32,000 psi, or 28,000 psi), to produce the emulsion. The resulting emulsion may be sterilized by, e.g., filtration through one or more suitable sterilizing filters, e.g., one or more 0.2 μm filters, and then filled into appropriate containers for pharmaceutical applications.

The active pharmaceutical ingredient (API) may include one or more active pharmaceutical ingredients (APIs) for which at least one co-emulsifier is needed to dissolve the API effectively in the oil phase, i.e., wherein dissolving the API in the oil phase of an emulsion effectively requires the aid of at least one co-emulsifier. APIs suitable for use in the process of the invention may include, for example, aprepitant, propofol, clevidipine, diazepam, etomidate, perfluorodecalin, perflurotripropy lamine, prostaglandins (e.g., alprostadil), corticosteroids (e.g., dexamethasone), flurbiprofen, lipids (e.g., long- and medium-chain triglycerides), fat soluble vitamins (e.g., A, D2, E, K1), and the like, and combinations thereof.

The active pharmaceutical ingredient (API) may include or exist in the form of particles, e.g., crystalline, partially crystalline, polycrystalline, polymorphic, and/or amorphous particles of the API. Particles of the active pharmaceutical ingredient (API) may be obtained by any suitable process known in the art for producing API particles such as, for example, micronization, which may include, e.g., passing the API through an appropriately mesh-sized sieve or screen before combining the API (as screened or sieved particles) with a solid emulsifier to produce the dry mixture containing the API and a solid emulsifier. For instance, particles of the active pharmaceutical ingredient (API) may be obtained by passing the API through a screen having a mesh size of 18-400, 50-400, 70-400, 100-400, 140-400, 170-400, 170-325, 170-270, or 170-230, e.g., a 200 mesh screen, and the resulting particles may thereafter be combined with a solid emulsifier to produce the dry mixture containing the API and a solid emulsifier. In some embodiments, particles of the active pharmaceutical ingredient (API) are obtained by passing the API through a 200 mesh screen, and the resulting API particles are combined with a solid emulsifier to produce the dry mixture, e.g., as in step (a) (mixing an active pharmaceutical ingredient (API) with a solid emulsifier to form a dry mixture). The particle size of the active pharmaceutical ingredient (API), before being combined with a solid emulsifier, may range, e.g., from about 0.01 μm to about 1000 μm, from about 0.01 μm to about 500 μm, from about 0.01 μm to about 400 μm, from about 0.01 μm to about 300 μm, from about 0.01 μm to about 200 μm, from about 0.01 μm to about 100 μm, from about 0.01 μm to about 50 μm, or from about 0.01 μm to about 30 μm. In some embodiments, the active pharmaceutical ingredient (API) has a particle size D90 of less than 100 μm, e.g., a Doo of less than 50 μm, a D90 of less than 30 μm, a D90 of less than 20 μm, or a D₉₀ of less than 10 μm, before or after being combined with a solid emulsifier to produce the dry mixture.

In some embodiments, the solid emulsifier used in the dry mixture containing the active pharmaceutical ingredient (API) and solid emulsifier is included in an amount effective to deagglomerate and/or to inhibit agglomeration of API particles in the dry mixture. Without wishing to be bound by any particular theory, it is believed that combining the solid emulsifier with API particles in an amount effective to deagglomerate and/or inhibit agglomeration of the API particles in the dry mixture may improve the ability of the API particles to dissolve effectively in an oil phase, thereby minimizing the amount of co-emulsifier needed to dissolve the API therein.

In certain embodiments, the dry mixture containing the API and solid emulsifier may include a portion of the total amount of solid emulsifier used in the emulsion, wherein the remainder of solid emulsifier may be added separately to the oil phase before or after adding the dry mixture thereto. For example, the dry mixture containing the API and solid emulsifier may include a portion of the total amount of solid emulsifier used in the emulsion, wherein the remainder of solid emulsifier is included (e.g., pre-mixed) in the first oil phase produced in step (b) (mixing an oil with a co-emulsifier to form a first oil phase, wherein the first oil phase further includes the remainder of solid emulsifier). Thus, the process may include, for example, including a portion of the total amount of solid emulsifier in the first oil phase produced in step (b) (mixing an oil a co-emulsifier to form a first oil phase, wherein the first oil phase further includes a portion of the solid emulsifier), and adding the remainder of solid emulsifier via the dry mixture containing the API and the remainder of solid emulsifier in step (c) (combining the dry mixture with the first oil phase to form a second oil phase). The dry mixture containing the API and solid emulsifier may include any suitable proportion of the total amount of solid emulsifier used in the emulsion. For example, the dry mixture containing the API and solid emulsifier may include, e.g., from about 5 wt. % to about 100 wt. %, from about 5 wt. % to about 75 wt. %, from about 5 wt. % to about 70 wt. %, from about 5 wt. % to about 60 wt. %, from about 5 wt. % to about 50 wt. %, from about 5 wt. % to about 40 wt. %, from about 5 wt. % to about 30 wt. %, from about 5 wt. % to about 20 wt. %, from about 5 wt. % to about 10 wt. %, from about 10 wt. % to about 70 wt. %, from about 10 wt. % to about 60 wt. %, from about 10 wt. % to about 50 wt. %, from about 10 wt. % to about 40 wt. %, from about 10 wt. % to about 30 wt. %, from about 10 wt. % to about 20 wt. %, from about 20 wt. % to about 70 wt. %, from about 20 wt. % to about 60 wt. %, from about 20 wt. % to about 50) wt. %, from about 20 wt. % to about 40 wt. %, or from about 20) wt. % to about 30 wt. %, of the total amount of solid emulsifier used in the emulsion. In some embodiments, the dry mixture containing the API and solid emulsifier may include about 25 wt. % of the total amount of solid emulsifier used in the emulsion. The remainder of solid emulsifier may be included in the oil phase, e.g., in the first oil phase produced in step (b) (mixing an oil with a co-emulsifier to form a first oil phase, wherein the first oil phase further includes the remainder of solid emulsifier).

The proportion of solid emulsifier relative to the API in the dry mixture may vary depending the nature of API particles and solid emulsifier used for preparing the emulsion, taking into account variables such as, e.g., relative solubility of the API in a particular oil phase, particle size, particle surface properties, e.g., porosity, degree of crystallinity, etc., as well as properties associated with solid surfactant, e.g., the ability of the solid surfactant to sufficiently coat particles of a particular API, binding affinity of the solid surfactant for the API particles, ability of the solid surfactant to promote dissolution of the API particles in the oil phase, etc. If desired, the proportion of solid emulsifier relative to the API in the dry mixture may exceed the amount of solid emulsifier minimally required to deagglomerate and/or inhibit agglomeration of API particles in the dry mixture.

The solid emulsifier is preferably included in the emulsion an amount effective to promote emulsification of oil and aqueous phases sufficiently to provide a stable emulsion. e.g., by inhibiting flocculation, creaming, and/or coalescence (breaking up) of oil phase droplets of in an aqueous medium. The total amount of solid emulsifier used in the emulsion may range, for example, from about 1 wt. % to about 25 wt. %, from about 5 wt. % to about 20 wt. %, from about 8 wt. % to about 18 wt. %, from about 10 wt. % to about 18 wt. %, or from about 12 wt. % to about 16 wt. % of solid emulsifier relative to the total amount (weight/mass) of the emulsion. In some embodiments, the total amount of solid emulsifier used in the emulsion is about 10-15 wt. % (e.g., about 14 wt. %) solid emulsifier relative to the total amount (weight/mass) of emulsion.

The co-emulsifier is preferably included in the emulsion in an amount effective to promote effective dissolution of the API in the oil phase, and if needed, emulsification of distinct phases and/or emulsion stability. The total amount of co-emulsifier used in the emulsion may range, for example, from about 0.1 wt. % to about 15 wt. %, from about 1 wt. % to about 10 wt. %, from about 2 wt. % to about 8 wt. %, from about 3 wt. % to about 5 wt. %, or from about 2.5 wt. % to about 3.5 wt. % of co-emulsifier relative to the total amount (weight/mass) of the emulsion. In some embodiments, the total amount of co-emulsifier used in the emulsion is about 3 wt. % (e.g., about 2.9-3.0 wt. %) co-emulsifier relative to the total amount (weight/mass) of the emulsion.

In some embodiments, when a volatile co-emulsifier (e.g., ethanol) is used, it may be desirable to add a slight overage of co-emulsifier in the oil phase, for example, in step (b) (mixing an oil with a co-emulsifier to form a first oil phase), e.g., to compensate for evaporation of the co-emulsifier that may occur during the process prior to forming the final emulsion. If an overage of volatile co-emulsifier (e.g., ethanol) is used, the total amount of co-emulsifier used in the process is preferably no more than about 10% greater than the total amount of co-emulsifier contained in the final product after emulsification. In some embodiments, the process may include adding an overage of from about 1 wt. % to about 10 wt. % volatile co-emulsifier, e.g., adding a volatile co-emulsifier (e.g., ethanol) in an amount of from about 1 wt. % to about 10 wt. % greater than the total amount of co-emulsifier present in the final product after emulsification. For example, the process may include adding an overage of from about 2 wt. % to about 8 wt. % volatile co-emulsifier, e.g., adding a volatile co-emulsifier (e.g., ethanol) in an amount of from about 2 wt. % to about 8 wt. % greater than the amount of co-emulsifier present in the final product after emulsification. In some embodiments, the process may include adding an overage of about 5 wt. % volatile co-emulsifier, e.g., adding a volatile co-emulsifier (e.g., ethanol) in an amount of about 5 wt. % greater than the amount of co-emulsifier present in the final product after emulsification.

The solid emulsifier may include one or more pharmaceutically acceptable emulsifiers that exist in a solid or semi-solid state at ambient temperature, and which promotes emulsification of oil and aqueous phases sufficiently to provide a stable emulsion, e.g., by inhibiting flocculation, creaming, and/or coalescence (breaking up) of oil phase droplets of in an aqueous medium. The solid emulsifier may include, for example, one or more solid anionic emulsifiers (e.g., laurate salts such as, e.g., sodium laurate, potassium laurate, etc., triethanolamine stearate, sodium lauryl sulfate, alkyl polyoxyethylene sulfates, sodium dodecyl sulfate, dioctyl sodium sulfosuccinate), one or more solid nonionic emulsifiers (e.g., polyoxyethylene fatty acid derivatives of sorbitan esters (e.g., Tween emulsifiers), polyoxyethylene fatty alcohol ethers, sorbitan fatty acid esters, polyoxyethylene alkyl ethers (macrogols), polyoxyethylene sorbitan fatty acid esters, polyoxyethylene polyoxy propylene block copolymers (poloxamers), polyethylene glycol 400 monostearate, lanolin alcohols, ethoxylated lanolin, etc.), one or more solid cationic emulsifiers (e.g., one or more solid quaternary ammonium emulsifiers), one or more solid amphiphilic emulsifiers (e.g., one or more phospholipids such as, e.g., lecithin, e.g., egg yolk lecithin, mixtures of gly cerophospholipids, e.g., phosphatidylcholine, phosphatidylethanolamine, phosphatidylinositol, phosphatidylserine, and phosphatidic acid, etc.), and the like, and combinations thereof. Preferably, the solid emulsifier is suitable for use in injectable pharmaceuticals. In some embodiments, the solid emulsifier used in preparing the emulsion includes an egg lecithin, e.g., egg yolk lecithin.

The oil used in the oil phase of the emulsion may include one or more oils suitable for use in pharmaceutical emulsions. Pharmaceutically acceptable oils may include, for example, purified natural oils such as, for example, pharmaceutical grade soy bean oil, pharmaceutical grade safflower oil, pharmaceutical grade olive oil, pharmaceutical grade sesame oil, pharmaceutical grade fish oil, and the like, and combinations thereof. Preferably, the oil used in the oil phase is suitable for use in injectable pharmaceuticals. In some embodiments, the oil used in the oil phase includes soy bean oil.

The co-emulsifier used in the emulsion may include one or more co-emulsifiers that are suitable for use in pharmaceutical emulsions. In some embodiments, the co-emulsifier used in the emulsion may include one of more volatile co-emulsifiers such as, for example, ethanol, isopropyl alcohol, butanol, propylene glycol, glycofurol, isosorbide dimethyl ether, and the like, and combinations thereof. Preferably, the co-emulsifier used in the emulsion is suitable for use in injectable pharmaceuticals, and more preferably includes a volatile co-emulsifier suitable for use in injectable pharmaceuticals. In some embodiments, the co-emulsifier used in the emulsion includes ethanol.

The water used in the emulsion may include any water of a grade that is suitable for use in pharmaceutical emulsions. Pharmaceutically acceptable grades of water may include, for example USP purified water, USP water for injection (WFI). USP sterile water for injection, LUSP sterile water for inhalation, USP bacteriostatic water for injection, USP sterile water for irrigation, and the like, and combinations thereof. Preferably, the water used in the aqueous phase of emulsion is suitable for use in injectable pharmaceuticals. In some embodiments, the aqueous phase of the emulsion includes water for injection (WFI), e.g., USP water for injection.

The tonicity agent used in the emulsion may include one or more tonicity agents that are suitable for use in pharmaceutical emulsions. Pharmaceutically acceptable tonicity agents may include, for example, sucrose, dextrose, glycerin, mannitol, potassium chloride, and sodium chloride, and the like, and combinations thereof. Preferably, the tonicity agent used in the emulsion is suitable for use in injectable pharmaceuticals. In some embodiments, the tonicity agent used in the emulsion includes sucrose.

The pH modifier used in the emulsion may include one or more pH modifiers that are suitable for use in pharmaceutical emulsions. Pharmaceutically acceptable pH modifiers may include, for example, one or more pharmaceutically acceptable acids (e.g., HCl, acetic acid, citric acid, tartaric acid, etc.), one or more pharmaceutically acceptable bases (e.g., NaOH, pharmaceutically acceptable amines, e.g., ammonium hydroxide, etc.), one or more pharmaceutically acceptable buffers (e.g., acetate buffers, citrate buffers, phosphate buffers, etc.), one or more pharmaceutically acceptable fatty acid acids or salts thereof (e.g., oleic acid, steric acid, lauric acid, linoleic acid, linolenic acid, palmitic acid, myristic acid, etc., and/or one or more pharmaceutically acceptable salts thereof), and the like, and combinations thereof. Preferably, the pH modifier used in the emulsion is suitable for use in injectable pharmaceuticals. In some embodiments, the pH modifier used in the emulsion includes oleic acid or a pharmaceutically acceptable salt thereof, e.g., sodium oleate.

In one embodiment, the invention provides a process for preparing an emulsion, which process includes emulsifying (i) an oil phase containing an oil, an active pharmaceutical ingredient (API) which includes aprepitant, a solid emulsifier which includes an egg yolk lecithin, and a co-emulsifier which includes ethanol, and (ii) an aqueous phase containing water, a tonicity agent which includes sucrose, and a pH modifier which includes oleic acid or a salt thereof, wherein the ethanol is present at a pharmaceutically acceptable level following emulsification without removing (e.g., evaporating) ethanol from the emulsion. The process includes combining a dry mixture containing aprepitant and at least a portion of an egg yolk lecithin (e.g., adding the aprepitant as a pre-mixed powder containing the aprepitant and egg yolk lecithin), with an oil, ethanol, and, optionally, an additional quantity of egg yolk lecithin, to produce the oil phase, combining the oil phase with an aqueous phase containing water, sucrose, and oleic acid or a salt thereof, and homogenizing the oil and aqueous phases to produce a pharmaceutically acceptable emulsion containing a pharmaceutically acceptable level of ethanol following emulsification without a step of removing (e.g., evaporating) ethanol from the emulsion.

In some embodiments, the invention provides a process for producing a pharmaceutically acceptable aprepitant emulsion, which process includes (a) mixing aprepitant with an egg yolk lecithin to form a dry mixture, (b) mixing an oil with ethanol to form a first oil phase. (c) combining the dry mixture with the first oil phase to form a second oil phase. (d) combining water, sucrose, and oleic acid or a salt thereof to form an aqueous phase, and (e) homogenizing the second oil phase with the aqueous phase to form the emulsion, without removing (e.g., without evaporating) ethanol following homogenization.

The dry mixture produced in step (a) (mixing aprepitant with an egg yolk lecithin to form a dry mixture) may be prepared by any method suitable for combining the aprepitant with the egg yolk lecithin, e.g., by dry mixing the aprepitant with the egg yolk lecithin. For example, the dry mixture may be produced by combining the aprepitant and a suitable quantity of egg yolk lecithin in a dry (e.g., solid) state, and mechanically agitating the aprepitant and egg yolk lecithin together as dry components, e.g., in a mixing bag or other suitable vessel, sufficiently to mix the components.

The first oil phase produced step (b) (mixing an oil with ethanol to form a first oil phase) may be prepared by methods that are known in the art. For example, the first oil phase may be produced by combining the oil and ethanol and, optionally, further including a portion of the egg yolk lecithin, in a suitable compounding tank, and mixing the components. e.g., at ambient temperature, until the components are dissolved.

The second oil phase produced in step (c) (combining the dry mixture with the first oil phase to form a second oil phase) may be prepared by methods that are known in the art. For example, the second oil phase may be produced by adding the dry mixture containing aprepitant and egg yolk lecithin to the first oil phase while mixing in a suitable compounding tank, mixing the components, e.g., at ambient temperature, and optionally warming the mixture (e.g., at about 55-65° C.) while mixing until the dry mixture is dissolved. In some embodiments, the process for producing the second oil phase, e.g., as in step (c), further includes applying a vacuum the second oil phase to evacuate the second oil phase. e.g., sufficiently to remove at least a substantial portion of oxygen therefrom, backfilling the evacuated second oil phase with an inert gas, e.g., nitrogen, and then heating the second oil phase in an atmosphere of the inert gas until the dry mixture completely dissolves therein. For example, the second oil phase may be produced by combining the dry mixture containing aprepitant and egg yolk lecithin with the first oil phase in a suitable vessel, e.g., a compounding tank, applying a vacuum to the vessel to evacuate the vessel, backfilling with the evacuated vessel with nitrogen, and then, if needed, heating the vessel in a nitrogen atmosphere until complete dissolution of the dry mixture in the second oil phase.

The aqueous phase produced in step (d) (combining water, sucrose, and oleic acid or a salt thereof to form an aqueous phase) may be prepared by methods that are known in the art. For example, the aqueous phase may be produced by adding appropriate amounts of sucrose and oleic acid or salt thereof to a suitable compounding tank containing an appropriate volume of water, e.g., water for injection, and mixing the components, e.g., at ambient temperature or above (e.g., at about 40-50° C.) until the sucrose and oleic acid or salt thereof are dissolved.

The emulsion produced in step (e) (homogenizing the second oil phase with the aqueous phase to form the emulsion) may be prepared by homogenizing the second oil phase and aqueous phase using methods that are known in the art. For example, the emulsification process may be carried out by transferring the second oil phase to the aqueous phase contained in a suitable compound tank while mixing at low speed (e.g., 350 rpm), followed by homogenization of the oil and water phases at high speed (e.g., 2500 rpm), and then microfluidization of the oil and water phases (e.g., at 28,000 psi), to produce the emulsion. The resulting emulsion may be sterilized by, e.g., filtration through one or more suitable sterilizing filters, e.g., one or more 0.2 μm filters, and then filled into appropriate containers for pharmaceutical applications.

The aprepitant may include or exist in the form of particles, e.g., crystalline, partially crystalline, polycrystalline, polymorphic, and/or amorphous particles of aprepitant. Particles of aprepitant may be obtained by any suitable process known in the art for producing API particles such as, for example, micronization, which may include, e.g., passing the aprepitant through an appropriately mesh-sized sieve or screen before combining the aprepitant (as screened or sieved particles) with an egg yolk lecithin to produce the dry mixture containing the aprepitant egg yolk lecithin. For instance, particles of aprepitant may be obtained by passing the aprepitant through a screen having a mesh size of 18-400, 50-400, 70-400, 100-400, 140-400, 170-400, 170-325, 170-270, or 170-230, e.g., a 200 mesh screen, and the resulting particles may thereafter be combined with an egg yolk lecithin to produce the dry mixture containing the aprepitant and egg yolk lecithin. In some embodiments, particles of aprepitant are obtained by passing the aprepitant through a 200 mesh screen, and the resulting aprepitant particles are thereafter combined with an egg yolk lecithin to produce the dry mixture, e.g., as in step (a) (mixing aprepitant with an egg yolk lecithin to form a dry mixture). The particle size of the aprepitant, before being combined with egg yolk lecithin, may range, e.g., from about 0.01 μm to about 1000 μm, from about 0.01 μm to about 500 μm, from about 0.01 μm to about 400 μm, from about 0.01 μm to about 300 μm, from about 0.01 μm to about 200 μm, from about 0.01 μm to about 100 μm, from about 0.01 μm to about 50 μm, or from about 0.01 μm to about 30 μm. In some embodiments, the aprepitant has a particle size Doo of less than 100 μm, e.g., a D₉₀ of less than 50 μm, a D₉₀ of less than 30 μm, a D₉₀ of less than 20 μm, or a D₉₀ of less than 10 μm, before or after being combined with egg yolk lecithin to produce the dry mixture.

The aprepitant used for preparing the emulsion may include particles of crystalline aprepitant. For instance, the aprepitant may include particles containing one or more crystalline polymorphs of aprepitant such as, for example, particles containing a combination of two or more crystalline polymorphs of aprepitant. In some embodiments, the aprepitant used for preparing the emulsion includes particles containing crystalline polymorph Form I of aprepitant, crystalline polymorph Form II of aprepitant, or a mixture of crystalline polymorphs Form I and Form II of aprepitant. In some embodiments, the aprepitant used for preparing the emulsion includes particles containing a mixture of polymorph Form I and polymorph Form II of aprepitant. Polymorphic Forms I and II of aprepitant are described, for example, in U.S. Pat. Nos. 6,229,010, and 9.227,958, and in Helmy et al., Characterization and Quantitation of Aprepitant Drug Substance Polymorphs by Attenuated Total Reflectance Fourier Transform Infrared Spectroscopy, Anal. Chem. 2003, 75:605-611.

In some embodiments, the aprepitant used for preparing the emulsion includes a mixture of polymorph Form I and polymorph Form II of aprepitant, wherein the amount of polymorph Form I constitutes not more than about 80 wt. % of the aprepitant (e.g., aprepitant particles which include at most about 80 wt. % of the Form I polymorph). The process of the invention also includes embodiments in which the aprepitant used for preparing the emulsion includes a mixture of polymorph Form I and polymorph Form II of aprepitant, wherein the amount of polymorph Form II is not less than about 20 wt. % of the aprepitant (e.g., aprepitant particles which include at least about 20 wt. % of the Form II polymorph). The process of the invention further includes embodiments in which the aprepitant used for preparing the emulsion includes a mixture of polymorph Form I and polymorph Form II of aprepitant, wherein the amount of polymorph Form I is from about 70 wt. % to about 80 wt. % of the aprepitant (e.g., aprepitant particles of which about 70-80 wt. % are in the form of polymorph Form I) and the amount of polymorph Form II is from about 20 wt. % to about 30 wt. % of the aprepitant (e.g., aprepitant particles of which about 20-30 wt. % are in the form of polymorph Form II).

The process of the invention also includes embodiments in which the aprepitant is substantially free of amorphous aprepitant. The aprepitant used for preparing the emulsion thus may include, for example, particles containing one or more crystalline forms of aprepitant, wherein the particles are substantially free of amorphous aprepitant. Such particles may include, for example, particles containing a mixture of polymorph Form I and polymorph Form II of aprepitant, wherein the particles are substantially free of amorphous aprepitant. Such particles also may include, for example, particles containing a mixture of polymorph Form I and polymorph Form II of aprepitant, wherein the amount of polymorph Form I constitutes not more than (i.e., at most) about 80% (wt/wt) of the aprepitant, and wherein the particles are substantially free of amorphous aprepitant. Such particles additionally may include, for example, aprepitant in the form of a mixture of polymorph Form I and polymorph Form II of aprepitant, wherein the amount of polymorph Form II is not less than (i.e., at least) about 20% (wt/wt) of the aprepitant, and wherein the particles are substantially free of amorphous aprepitant. The process of the invention further includes embodiments in which the aprepitant used for preparing the emulsion includes particles containing a mixture of polymorph Form I and polymorph Form II of aprepitant, wherein the amount of polymorph Form I is about 70%-80% (wt/wt) of the aprepitant (e.g., about 70-80 wt. % of the aprepitant in the particles is of polymorph Form I), the amount of polymorph Form II is about 20%-30% (wt/wt) of the aprepitant (e.g., about 20-30 wt. % of the aprepitant in the particles is of polymorph Form II), and the particles are substantially free of amorphous aprepitant.

In some embodiments, the egg yolk lecithin used in the dry mixture containing the aprepitant and egg yolk lecithin is included in an amount effective to deagglomerate and/or to inhibit agglomeration of aprepitant particles in the dry mixture. Without wishing to be bound by any particular theory, it is believed that combining the egg yolk lecithin with aprepitant particles, in an amount effective to deagglomerate and/or inhibit agglomeration of particles in the dry mixture, may improve the ability of the aprepitant to dissolve effectively in the oil phase, thereby minimizing the amount of ethanol needed to dissolve the aprepitant therein.

In certain embodiments, the dry mixture containing aprepitant and egg yolk lecithin, e.g., as produced in step (a) (mixing aprepitant with an egg yolk lecithin to form a dry mixture), may include a portion of the total amount of egg yolk lecithin used in the emulsion, wherein the remainder of egg yolk lecithin may be added separately to the oil phase before or after adding the dry mixture to the oil phase. For example, the dry mixture containing aprepitant and egg yolk lecithin (e.g., as produced in step (a)) may include a portion of the total amount of egg yolk lecithin used in the emulsion, wherein the remainder of egg yolk lecithin is included (e.g., pre-mixed) in the first oil phase produced in step (b) (mixing an oil with ethanol to form a first oil phase, wherein the first oil phase further includes the remainder of egg yolk lecithin). Thus, the process may include, for example, including a portion of the total amount of egg yolk lecithin in the first oil phase produced in step (b) (mixing an oil with ethanol to form a first oil phase, wherein the first oil phase further includes egg yolk lecithin), and adding the remainder of egg yolk lecithin via the dry mixture containing aprepitant and the remainder of egg yolk lecithin in step (c) (combining the dry mixture with the first oil phase to form a second oil phase). The dry mixture containing aprepitant and egg yolk lecithin may include any suitable proportion of the total amount of egg yolk lecithin used in the emulsion. For example, the dry mixture containing aprepitant and egg yolk lecithin may include, e.g., from about 5 wt. % to about 100 wt. %, from about 5 wt. % to about 75 wt. %, from about 5 wt. % to about 70 wt. %, from about 5 wt. % to about 60 wt. %, from about 5 wt. % to about 50 wt. %, from about 5 wt. % to about 40 wt. %, from about 5 wt. % to about 30 wt. %, from about 5 wt. % to about 20 wt. %, from about 5 wt. % to about 10 wt. %, from about 10 wt. % to about 70 wt. %, from about 10 wt. % to about 60 wt. %, from about 10 wt. % to about 50 wt. %, from about 10 wt. % to about 40 wt. %, from about 10 wt. % to about 30 wt. %, from about 10 wt. % to about 20 wt. %, from about 20 wt. % to about 70 wt. %, from about 20 wt. % to about 60 wt. %, from about 20 wt. % to about 50 wt. %, from about 20 wt. % to about 40 wt. %, or from about 20 wt. % to about 30 wt. %, of the total amount of the egg yolk lecithin used in the emulsion. In some embodiments, the dry mixture containing aprepitant and egg yolk lecithin, e.g., as produced in step (a) (mixing aprepitant with an egg yolk lecithin to form a dry mixture), may include about 25 wt. % of the total amount of egg yolk lecithin used in the emulsion. The remainder of egg yolk lecithin may be included in the oil phase, e.g., in the first oil phase produced in step (b) (mixing an oil with ethanol to form a first oil phase, wherein the first oil phase further includes the remainder of egg yolk lecithin).

The proportion of egg yolk lecithin relative to aprepitant in the dry mixture may vary based on the nature of the aprepitant particles and egg yolk lecithin used for preparing the emulsion, taking into account variables such as, e.g., relative solubility of the aprepitant in the oil phase, particle size, degree of crystallinity, polymorphism, particle surface properties, e.g., porosity, etc., as well as properties associated with the egg yolk lecithin, e.g., the ability of the lecithin to sufficiently coat and/or bind to particles of aprepitant, the ability of the lecithin to promote dissolution of the aprepitant particles in the oil phase, etc. If desired, the proportion of egg yolk lecithin relative to aprepitant in the dry mixture may exceed the amount of egg yolk lecithin minimally required to deagglomerate and/or inhibit agglomeration of aprepitant particles in the dry mixture.

The egg yolk lecithin is preferably included in the emulsion an amount effective to promote emulsification of the oil and aqueous phases sufficiently to provide a stable emulsion, e.g., by inhibiting flocculation, creaming, and/or coalescence (breaking up) of oil phase droplets of in an aqueous medium. The total amount of egg yolk lecithin used in the emulsion may range, for example, from about 1 wt. % to about 25 wt. %, from about 5 wt. % to about 20 wt. %, from about 8 wt. % to about 18 wt. %, from about 10 wt. % to about 18 wt. %, or from about 12 wt. % to about 16 wt. % of egg yolk lecithin relative to the total amount (weight/mass) of the emulsion. In some embodiments, the total amount of egg yolk lecithin used in the emulsion is about 10-15 wt. % (e.g., about 14 wt. %) egg yolk lecithin relative to the total amount (weight/mass) of emulsion.

The ethanol is preferably included in the emulsion in an amount effective to promote effective dissolution of the aprepitant in the oil phase. The total amount of ethanol used in the emulsion may range, for example, from about 0.1 wt. % to about 10 wt. %, from about 1 wt. % to about 8 wt. %, from about 2 wt. % to about 5 wt. %, from about 2.5 wt. % to about 3.5 wt. %, or from about 2.75 wt. % to about 3.25 wt. % of ethanol relative to the total amount (weight/mass) of the emulsion. In some embodiments, the total amount of ethanol used in the emulsion is about 3 wt. % (e.g., about 2.9-3.0 wt. %) ethanol relative to the total amount (weight/mass) of the emulsion.

In some embodiments, when ethanol is used as a co-emulsifier, it may be desirable to add a slight overage of ethanol in the oil phase, for example, in step (b) (mixing an oil with ethanol to form a first oil phase), e.g., to compensate for ethanol evaporation that may occur during the process prior to forming the final emulsion. If an overage of ethanol is used, the total amount of ethanol used in the process is preferably no more than about 10% greater than the total amount of ethanol contained in the final product after emulsification. In some embodiments, the process may include adding an overage of from about 1 wt. % to about 10 wt. % ethanol, e.g., adding ethanol in an amount of from about 1 wt. % to about 10) wt. % greater than the total amount of ethanol present in the final product after emulsification. For example, the process may include adding an overage of from about 2% to about 8% ethanol, e.g., adding ethanol in an amount of from about 2 wt. % to about 8 wt. % greater than the amount of ethanol present in the final product after emulsification. In some embodiments, the process may include adding an overage of about 5 wt. % ethanol, e.g., adding ethanol in an amount of about 5 wt. % greater than the amount of ethanol present in the final product after emulsification.

The egg yolk lecithin may include one or more pharmaceutically acceptable grades of egg yolk lecithin. Preferably, the grade of egg yolk lecithin used for preparing the emulsion is suitable for use in injectable pharmaceuticals. In some embodiments, the egg yolk lecithin used in preparing the emulsion may include, for example, any egg yolk lecithin that complies with Egg Phospholipid NF monograph, such as, e.g., Lipoid E 80.

The oil used for the oil phase of the emulsion may include one or more oils suitable for use in pharmaceutical emulsions. Pharmaceutically acceptable oils may include, for example, purified natural oils such as, for example, pharmaceutical grade soy bean oil, pharmaceutical grade safflower oil, pharmaceutical grade olive oil, pharmaceutical grade sesame oil, pharmaceutical grade fish oil, and the like, and combinations thereof. Preferably, the oil used in the oil phase is suitable for use in injectable pharmaceuticals. In some embodiments, the oil used in the oil phase includes soy bean oil.

The ethanol used as a co-emulsifier in the emulsion may include one or more grades of ethanol that are suitable for use in pharmaceutical emulsions. Preferably, the ethanol used in the emulsion is of a grade that is suitable for use in injectable pharmaceuticals. In some embodiments, the ethanol used in the emulsion includes Dehydrated Alcohol USP, Alcohol USP or Ethanol USP/Ph. Eur.

The water used for the aqueous phase of the emulsion may include any water of a grade that is suitable for use in pharmaceutical emulsions. Pharmaceutically acceptable grades of water may include, for example, USP purified water, USP water for injection (WFI), USP sterile water for injection, LUSP sterile water for inhalation, USP bacteriostatic water for injection, USP sterile water for irrigation, and the like, and combinations thereof. Preferably, the water used in the aqueous phase of emulsion is suitable for use in injectable pharmaceuticals. In some embodiments, the aqueous phase of the emulsion includes water for injection (WFI), e.g., USP water for injection.

The sucrose used as a tonicity agent in the emulsion may include one or more grades of sucrose that are suitable for use in pharmaceutical emulsions. Preferably, the sucrose used in the emulsion is of a grade that is suitable for use in injectable pharmaceuticals. In some embodiments, the sucrose used in the emulsion includes Sucrose USP or Sucrose NF/Ph. Eur.

The oleic acid or salt thereof, which is used as a pH modifier in the emulsion, may include one or more grades of oleic acid and/or oleic acid salts that are suitable for use in pharmaceutical emulsions. Preferably, the oleic acid or salt thereof used in the emulsion is suitable for use in injectable pharmaceuticals. In some embodiments, the pH modifier used in the emulsion includes sodium oleate.

The invention further provides an emulsion produced by the process disclosed herein. The emulsion produced by the process disclosed herein is stable, preferably for at least one year under refrigerated and/or room temperature conditions. As used herein, the terms “stable” and “stability” encompass any characteristic of the emulsion which may be affected by storage conditions including, without limitation, potency, total impurities. API degradation products, crystal content, ethanol content, mean globule size, percentage of fat residing in globules larger than 5 μm (PFAT5), zeta potential, dissolution, residual solvents, specific optical rotation, optical purity, appearance, viscosity, sterility, particulates (visible and subvisible) and color. The storage conditions which may affect stability include, for example, duration, temperature, humidity, and/or light exposure.

Methods for determining the stability of an emulsion of the invention with respect to a given parameter are well-known in the art. For example, API-related impurities may be assessed by high-performance liquid chromatography (HPLC) or thin layer chromatography (TLC), and ethanol or residual solvent content may be assessed by gas chromatography. Unless indicated otherwise, a percentage amount of any individual impurity or total impurities reported herein in the emulsion is determined by a peak area percent method using HPLC and percentage amount of ethanol content is determined by a peak area percent method using gas chromatography.

In some embodiments, the aprepitant emulsion produced by the process disclosed herein is stable for at least one year. For example, the aprepitant emulsion produced by the process disclosed herein may remain free of visible aprepitant crystals, e.g., will not contain or produce visible crystals of aprepitant, following storage of the emulsion under refrigerated temperature (e.g., 5° C. ±3° C./ambient humidity) and/or room temperature (e.g., 25° C. ±2° C./60% relative humidity (RH)+5% RH) conditions for at least about 3 months, e.g., at least about 6 months, at least about 9 months, at least about 1 year, at least about 18 months, or at least about 2 years. In some embodiments, the aprepitant emulsion produced by the process disclosed herein remains free of visible aprepitant crystals following storage of the emulsion under refrigerated temperature and/or room temperature conditions for 3 months or more, e.g., at least about 6 months, at least about 9 months, at least about 1 year, at least about 18 months, or at least about 2 years. In other embodiments, the aprepitant emulsion produced by the process disclosed herein contains about 2.5% or less (e.g., about 2.0% or less, about 1.5% or less, about 1.0% or less, about 0.75% or less, about 0.5% or less, or about 0.25% or less) of visible aprepitant crystals following storage of the emulsion under refrigerated temperature and/or room temperature conditions for at least about 3 months, e.g., at least about 6 months, at least about 9 months, at least about 1 year, at least about 18 months, or at least about 2 years. In still yet other embodiments, the aprepitant emulsion produced by the process disclosed herein contains about 0.1% to about 2.0% or any range therein (e.g., 0.1%-1.0%. 0).4%-0.8%. 0.2%-1.2%, 0.3%-0.9%. 0.1%-0.6%, 0.5%-1.5%, or 0.2%-0.5%) of visible aprepitant crystals following storage of the emulsion under refrigerated temperature and/or room temperature conditions for at least about 3 months, e.g., at least about 6 months, at least about 9 months, at least about 1 year, at least about 18 months, or at least about 2 years.

In some embodiments, the emulsion produced by the process disclosed herein contains about 1% or less (e.g., about 0).8% or less, about 0.6% or less, about 0.4% or less, about 0.3% or less, or about 0.2% or less) of total aprepitant-related impurities following storage of the emulsion under refrigerated temperature and/or room temperature conditions for at least about 3 months, e.g., at least about 6 months, at least about 9 months, at least about 1 year, at least about 18 months, or at least about 2 years. In other embodiments, the aprepitant emulsion produced by the process disclosed herein contains about 0.1% to about 1.0% or any range therein (e.g., 0.1%-0.8%, 0.2%-0.8%, 0.3%-0.9%, 0.1%-0.4%, 0.2%-0.6%, 0.2%-0.5%, or 0.3%-0.7%) of total aprepitant-related impurities following storage of the emulsion under refrigerated temperature and/or room temperature conditions for at least about 3 months, e.g., at least about 6 months, at least about 9 months, at least about 1 year, at least about 18 months, or at least about 2 years.

In certain embodiments, the emulsion produced by the process disclosed herein contains about 0.5% or less (e.g., about 0.4% or less, about 0.3% or less, about 0.2% or less, about 0.1% or less, or about 0.05% or less) of aprepitant N-oxide following storage of the emulsion under refrigerated temperature and/or room temperature conditions for at least about 3 months, e.g., at least about 6 months, at least about 9 months, at least about 1 year, at least about 18 months, or at least about 2 years. In other embodiments, the aprepitant emulsion produced by the process disclosed herein contains about 0.05% to about 0.5% or any range therein (e.g., 0.05%-0.4%. 0.05%-0.2%, 0.05%-0.3%, 0.1%-0.15%, 0.1%-0.2%, 0.1%-0.3%, or 0.15%-0.25%) of aprepitant N-oxide as determined by following storage of the emulsion under refrigerated temperature and/or room temperature conditions for at least about 3 months, e.g., at least about 6 months, at least about 9 months, at least about 1 year, at least about 18 months, or at least about 2 years.

In some embodiments, the emulsion produced by the process disclosed herein contains about 90% or more, e.g., about 92% or more, about 93% or more, about 94% or more, about 95% or more, about 96% or more, or about 97% or more, of the labeled concentration of aprepitant after storage under refrigerated temperature and/or room temperature conditions for at least about 3 months, e.g., at least about 6 months, at least about 9 months, at least about 1 year, at least about 18 months, or at least about 2 years. In other embodiments, the emulsion produced by the process disclosed herein contains about 110% or less, e.g., about 105% or less, about 104% or less, about 103% or less, about 102% or less, or about 101% or less, of the of the labeled concentration of aprepitant after storage under refrigerated temperature and/or room temperature conditions for at least about 3 months, e.g., at least about 6 months, at least about 9 months, at least about 1 year, at least about 18 months, or at least about 2 years. In yet other embodiments, the emulsion produced by the process disclosed herein contains from about 93% to about 103% (e.g., 93%-102%, 93%-101%. 94%-102%. 94%-101%. 95%-102%, or 95%-101%.), of the labeled concentration of aprepitant after storage under refrigerated temperature and/or room temperature conditions for at least about 3 months, e.g., at least about 6 months, at least about 9 months, at least about 1 year, at least about 18 months, or at least about 2 years.

In certain embodiments, the emulsion produced by the process disclosed herein contains about 90% or more, e.g., about 95% or more, about 96% or more, about 97% or more, about 98% or more, or about 99% or more of the labeled concentration of ethanol after storage under refrigerated temperature and/or room temperature conditions for at least about 3 months, e.g., at least about 6 months, at least about 9 months, at least about 1 year, at least about 18 months, or at least about 2 years. In other embodiments, the emulsion produced by the process disclosed herein contains about 110% or less, e.g., about 105% or less, about 104% or less, about 103% or less, or about 102% or less, of the of the labeled concentration of ethanol after storage under refrigerated temperature and/or room temperature conditions for at least about 3 months, e.g., at least about 6 months, at least about 9 months, at least about 1 year, at least about 18 months, or at least about 2 years. In yet other embodiments, the emulsion produced by the process disclosed herein contains from about 95% to about 105% (e.g., 96%-104%, 96%-103%, 97%-104%, 97%-103%, 98%-104%, or 98%-103%), of the labeled concentration of ethanol after storage under refrigerated temperature and/or room temperature conditions for at least about 3 months, e.g., at least about 6 months, at least about 9 months, at least about 1 year, at least about 18 months, or at least about 2 years.

It surprisingly has been discovered that an emulsion produced according to the invention has less variation (e.g., lot-to-lot and/or vial-to-vial) in particular physiochemical properties as compared the commercially available CINVANTI™ (aprepitant) emulsion.

In some embodiments, the emulsion produced by the process disclosed herein contains less variation in aprepitant dissolution as compared to the commercially available CINVANTI™ (aprepitant) emulsion. In certain embodiments, the emulsion produced by the process disclosed herein releases about 64% or more, e.g., about 66% or more, about 68% or more, about 70% or more, about 71% or more, about 72% or more, about 73% or more, about 74% or more, or about 75% or more, of the aprepitant present in the emulsion within 5 minutes in a USP apparatus IV flow through cell dissolution tester based on the pooled mean of multiple samples obtained from different vials of multiple manufacturing lots (e.g., 12 vial samples from each of 3 lots). In other embodiments, the emulsion produced by the process disclosed herein releases about 88% or less, e.g., about 86% or less, about 84% or less, about 82% or less, about 80% or less, about 79% or less, about 78% or less, or about 76% or less, of the aprepitant present in the emulsion within 5 minutes in a USP apparatus IV flow through cell dissolution tester based on the pooled mean of multiple samples obtained from different vials of multiple manufacturing lots (e.g., 12 vial samples from each of 3 lots). In yet other embodiments, the emulsion produced by the process disclosed herein releases about 64% to about 88%, e.g., about 64% to about 86%, about 68% to about 84%, about 70% to about 80%, about 71% to about 78%, about 71% to about 76%, or about 72% to about 74%, of the aprepitant present in the emulsion within 5 minutes in a USP apparatus IV flow through cell dissolution tester based on the pooled mean of multiple samples obtained from different vials of multiple manufacturing lots (e.g., 12 vial samples from each of 3 lots).

In certain embodiments, the emulsion produced by the process disclosed herein has a relative standard deviation (RSD) of aprepitant release of about 8% or less, e.g., 7.5% or less, 7.0% or less, 6.5% or less, 6.0% or less, 5.5% or less, 5.0% or less, or 4.5% or less, within 10 minutes in a USP apparatus IV flow through cell dissolution tester based on the pooled mean of multiple samples obtained from different vials of multiple manufacturing lots (e.g., 12 vial samples from each of 3 lots). In other embodiments, the emulsion produced by the process disclosed herein has an RSD of aprepitant release of about 2.0% or more, e.g., 2.5% or more, 3.0% or more, 3.5% or more, 4.0% or more, 4.5% or more, or 5.0% or more, within 10 minutes in a USP apparatus IV flow through cell dissolution tester based on the pooled mean of multiple samples obtained from different vials of multiple manufacturing lots (e.g., 12 vial samples from each of 3 lots). In yet other embodiments, the emulsion produced by the process disclosed herein has an RSD of aprepitant release of about 2.0% to about 8.0%, e.g., about 2.5% to about 7.5%, about 3.0% to about 7.0%, about 3.5% to about 7.5%, about 3.5% to about 6.5%, about 4.0% to about 6.0%, or about 4.0% to about 5.0%, within 10 minutes in a USP apparatus IV flow through cell dissolution tester based on the pooled mean of multiple samples obtained from different vials of multiple manufacturing lots (e.g., 12 vial samples from each of 3 lots).

In some embodiments, the emulsion produced by the process disclosed herein contains less variation in globule size distribution as compared to the commercially available CINVANTI™ (aprepitant) emulsion. In certain embodiments, the emulsion produced by the process disclosed herein has a SPAN ([D₉₀-D₁₀]/D₅₀ by laser diffraction) of about 0.70 or less, e.g., about 0.69 or less, about 0.68 or less, about 0.67 or less, about 0.66 or less, about 0.65 or less, about 0.64 or less, or about 0.63 or less, based on the pooled mean SPAN of multiple samples obtained from different vials of multiple manufacturing lots (e.g., 10 vial samples from each of 3 lots). In other embodiments, the emulsion produced by the process disclosed herein has a SPAN of about 0.48 or more, e.g., about 0.50 or more, about 0.52 or more, about 0.54 or more, about 0.56 or more, about 0.58 or more, about 0.60 or more, or about 0.62 or more, based on the pooled mean SPAN of multiple samples obtained from different vials of multiple manufacturing lots (e.g., 10 vial samples from each of 3 lots). In yet other embodiments, the emulsion produced by the process disclosed herein has a SPAN of about 0.48 to about 0.70, e.g., about 0.52 to about 0.70, about 0.56 to about 0.68, about 0.58 to about 0.68, about 0.60 to about 0.68, about 0.62 to about 0.67, or about 0.65 to about 0.67, based on the pooled mean SPAN of multiple samples obtained from different vials of multiple manufacturing lots (e.g., 10 vial samples from each of 3 lots).

In some embodiments, the emulsion produced by the process disclosed herein has a standard deviation (o) of SPAN of 0.22 or less, e.g., 0.20 or less, 0.18 or less, 0.16 or less, 0.14 or less, 0.12 or less, or 0.10 or less, based on the pooled mean SPAN of multiple samples obtained from different vials of multiple manufacturing lots (e.g., 10 vial samples from each of 3 lots). In other embodiments, the emulsion produced by the process disclosed herein has a standard deviation (o) of SPAN of 0.04 or more, e.g., 0.05 or more, 0.06 or more, 0.07 or more, 0.08 or more, 0.09 or more, or 0.10 or more, based on the pooled mean SPAN of multiple samples obtained from different vials of multiple manufacturing lots (e.g., 10 vial samples from each of 3 lots). In yet other embodiments, the emulsion produced by the process disclosed herein has a standard deviation (6) of SPAN of about 0.04 to about 0.22, e.g., about 0.06 to about 0.18, about 0.06 to about 0.16, about 0.08 to about 0.14, about 0.09 to about 0.12, or about 0.09 to about 0.11, based on the pooled mean SPAN of multiple samples obtained from different vials of multiple manufacturing lots (e.g., 10 vial samples from each of 3 lots).

The following examples further illustrate the invention but, of course, should not be construed as in any way limiting its scope.

### EXAMPLE 1

This example demonstrates an exemplary method of preparing an emulsion.

An aqueous phase is prepared by adding water (60 kg) to an aqueous phase tank and adjusted to a temperature of about 40° C.-50° C. While mixing, sucrose and sodium oleate are added to the aqueous phase tank, with additional water (up to a total of 83.3 kg) via flow meter. A temperature of about 40° C.-50° C., is maintained while mixing the contents of the aqueous phase tank until all added materials have been dissolved. After the aqueous phase is mixed, it is cooled to about 23° C.-27° C., and held at that temperature.

TABLE 1
Aqueous Phase Components

| Ingredient | Weight | |
|---|---|---|
| Water for injection | 83.3 | kg |
| Sucrose, NF | 6.75 | kg |
| Sodium oleate | 0.600 | kg |

An oil phase is prepared by preparing a dry pre-mixture of the active pharmaceutical ingredient (aprepitant) with egg lecithin. To prepare the dry pre-mix, egg lecithin (total 18.1 kg) is pre-weighed into four bags of equal weight, each being one quarter the total weight of lecithin to be used (i.e., about 4.53 kg each), and kept refrigerated until it is used. The active pharmaceutical ingredient, aprepitant, is pre-weighed (0.900 kg) into a separate weighing bag. A micronized aprepitant having a particle size D (90) below 10 μm and consisting of 55-80% polymorph Form I, 20-45% polymorph Form II is used to facilitate deagglomeration and dissolution. The pre-weighed aprepitant is then added to one of the four pre-weighed bags of lecithin, and mixed in the weighing bag by inverting the bag such that the micronized aprepitant is deagglomerated. Dehydrated alcohol (ethanol. 3.74 kg) and soybean oil (12.0 kg) are combined in the oil phase tank, and the dry pre-mixture of lecithin and aprepitant is added during agitation. An overage of not more than 5% the total weight of ethanol may be used during this step to ensure that aprepitant completely dissolves. To ensure that all of the aprepitant/lecithin mixture is dispensed from the bag, additional lecithin (from one of the other pre-weighed bags) is used to rinse the bag and is then added to the oil phase tank. During mixing, the remaining bags of lecithin are added to the oil phase tank. The oil phase tank is mixed until all solid materials therein are wetted, with little or no remaining dry powder in the oil phase tank. While continuing to mix, vacuum is applied to remove oxygen and nitrogen is backfilled into the oil phase tank, which is heated to 55° C.-65° C., such that, while mixing, the lecithin and API completely dissolve to form the oil phase.

TABLE 2
Oil Phase Components

| Ingredient | Weight | |
|---|---|---|
| Egg Phospholipids, NF (Egg Lecithin) | 18.1 | kg |
| Aprepitant (Active ingredient) | 0.900 | kg |
| Dehydrated Alcohol, USP (Ethanol) | 3.74 | kg |
| Soybean Oil, USP | 12.0 | kg |

Immediately following preparation of the oil phase, the oil phase is transferred into the aqueous phase tank and a crude emulsion is prepared. Prior to beginning the process of transferring the oil phase into the aqueous phase, the temperature of the aqueous phase tank is set to about 40° C. The oil phase is then transferred into the aqueous phase tank using the transfer pipe. The oil phase and aqueous phase are mixed at 350 rpm and then homogenized at 2500 rpm. After the oil phase has completely transferred, the oil phase tank is purged with nitrogen (N2) to expel any remaining oil phase from the transfer pipe. The mixed oil phase and aqueous phase are homogenized at 2500 rpm for an additional five (5) minutes after the purge of the transfer pipe, for a total homogenization time of about 10 minutes after the oil phase has completely transferred. The resulting crude emulsion is maintained at about 40° C., prior to and during the subsequent first cycle of the microfluidization step.

The crude emulsion is passed through a microfluidizer into a holding tank at 28,000 psi for a total of five (5) passes to produce a fine emulsion. Following the fifth microfluidizer cycle, the fine emulsion in a second compounding tank is maintained at a temperature of 2° C.-8° C. during the subsequent filtration step.

The resulting fine emulsion is filtered (0.2 μm) and then aseptically filled into sterilized vials under ambient air headspace. The filled vials are then stoppered, capped, and sealed prior to storage under refrigerated temperature or room temperature conditions.

### EXAMPLE 2

This example demonstrates the stability of an emulsion prepared according to the invention.

Three lots of aprepitant emulsion vials prepared as described in Example 1 were placed into stability chambers under refrigerated temperature (5° C. ±3° C./ambient humidity) or room temperature (25° C. ±2° C./60% relative humidity (RH)+5% RH) conditions. Following storage for 6 or 12 months, the emulsions were analyzed for aprepitant assay, aprepitant N-oxide, total impurities, crystal content, and ethanol content according to the following methods.

Samples for calculating aprepitant assay, aprepitant N-oxide and total impurities were prepared by transferring a portion of the exemplary aprepitant emulsion into a volumetric flask and diluting to volume with Diluent (water:acetonitrile:phosphoric acid 50:50:0.10) to produce a solution containing approximately 0.23 mg/mL aprepitant. A Standard Solution was prepared by dissolving aprepitant in Diluent to produce a solution containing approximately 0.23 mg/mL aprepitant. The samples were run on C18 HPLC using gradient elution, the chromatograms were recorded, and the amounts of aprepitant, aprepitant N-oxide, and total impurities were calculated by a peak area percent method. The results for the 3 exemplary lots are summarized in Tables 3 and 4.

TABLE 3
(6 months storage)

| | Lot 1 | | Lot 2 | | Lot 3 | |
|---|---|---|---|---|---|---|
| Parameter | 5° C. | 25° C. | 5° C. | 25° C. | 5° C. | 25° C. |
| Aprepitant Assay | 96.3% | 94.9% | 97.1% | 95.2% | 97.0% | 95.1% |
| Aprepitant N-oxide | <0.10% | <0.10% | <0.10% | <0.10% | <0.10% | <0.10% |
| Total Impurities | <0.10% | <0.10% | <0.10% | <0.10% | <0.10% | <0.10% |

TABLE 4
(12 months storage at 5° C.)

| Parameter | Lot 1 | Lot 2 | Lot 3 |
|---|---|---|---|
| Aprepitant Assay | 95.7% | 97.3% | 94.4% |
| Aprepitant N-oxide | <0.10% | <0.10% | <0.10% |
| Total Impurities | <0.10% | <0.10% | <0.10% |

Samples for calculating crystal content were prepared by transferring a portion of the exemplary aprepitant emulsion into thick-walled centrifuge tubes. The samples were subjected to ultracentrifugation at 80,000 RPM for 40-60 hours at 5-10° C., to yield products with gelatinous and oily layers on top with a nearly colorless aqueous layer at the bottom of the tube. The products were visually examined to determine whether a whitish solid precipitate/crystal deposit was observed at the bottom of the tube within the aqueous layer. If none, then the sample was documented as not having crystalline content.

If a precipitate was detected, the aqueous phase was collected by removing a portion of the upper gelatinous layer with a spatula, aspirating the organic liquid layer using a syringe with an 18-gauge needle, and collecting the bottom aqueous layer with a fresh syringe and needle. The precipitate was collected by cutting the ultracentrifuge tube horizontally with a pipe cutter to remove all the oily and gelatinous upper layers, and 0.5 mL of ethanol was repeatedly aspirated over the crystal deposit until all of the crystals were dissolved. The resulting solution was transferred to a 5-mL volumetric flask, diluted to volume with Diluent, and subjected to HPLC as described above. The quantitation limit for crystalline content was ≥0.0014% of the total aprepitant content in the emulsion.

None of the three exemplary lots of aprepitant emulsion vials prepared as described in Example 1 had crystalline content following storage for 6 or 12 months under refrigerated temperature or room temperature conditions.

Samples for calculating ethanol content were prepared by transferring a portion of the exemplary aprepitant emulsion into a volumetric flask, diluting to volume with Diluent (0.05% isopropanol in N-methyl-2-pyrrolidone (NMP)) and mixing to produce a solution containing approximately 0.275 mg/mL ethanol. A Standard Solution was prepared by mixing ethanol in NMP at a concentration of approximately 0.275 mg/mL ethanol. Portions of Diluent, Standard Solution, and exemplary aprepitant emulsion were transferred into headspace vials and run on a gas chromatography column (60 m×0.53 mm×3.0 μm). The chromatograms were recorded, the amount of ethanol was calculated using a peak area percent method, and then converted to percentage amount of initial ethanol content. The results for the 3 exemplary lots are summarized in Tables 5 and 6.

TABLE 5
(6 months storage)

| | Lot 1 | | Lot 2 | | Lot 3 | |
|---|---|---|---|---|---|---|
| | 5° C. | 25° C. | 5° C. | 25° C. | 5° C. | 25° C. |
| Ethanol | 99.6% | 99.1% | 99.5% | 99.1% | 98.8% | 99.0% |
| Content | | | | | | |

TABLE 6
(12 months storage at 5° C.)

| | Lot 1 | Lot 2 | Lot 3 |
|---|---|---|---|
| Ethanol Content | 99.0% | 98.6% | 98.8% |

The results of this example demonstrate that the aprepitant emulsion prepared according to the invention is stable with respect to multiple parameters during storage at refrigerated or room temperature conditions.

### EXAMPLE 3

This example demonstrates the dissolution of an emulsion of the invention as compared to the commercially available CINVANTI™ (aprepitant) emulsion.

Three lots of aprepitant emulsion vials prepared as described in Example 1 were placed into stability chambers under refrigerated temperature (5° C. ±3° C./ambient humidity) and stored for 6 months prior to analysis by a USP Apparatus IV dissolution method. Three lots of commercially available CINVANTI™ (aprepitant) emulsion that had been stored according to the package insert (refrigerated at 2° C.-8° C.) and were aged approximately 18 months (lots A and C) and 15 months (lot B) prior to expiry also were analyzed by the same dissolution method, as follows.

Samples containing approximately 7.2 mg/mL aprepitant were tested in a SOTAX CE7 Smart Dissolution Analyzer (closed system) using a flow rate of 4.0 mL/minute at a temperature of 37° C., in 200 mL of dissolution medium (35% v/v ethanol in phosphate buffered saline, pH 7.4). Samples were obtained at various timepoints and examined for aprepitant concentration by HPLC. The volumes and aprepitant concentration of solutions withdrawn at each sampling time were factored into calculations to determine the % aprepitant released, and the results for the exemplary emulsions and CINVANTI™ (aprepitant) emulsion are summarized in Tables 7 and 8, respectively.

TABLE 7

| Time | Lot 1 (n = 12) | | Lot 2 (n = 12) | | Lot 3 (n = 12) | | Pooled (n = 36) | |
|---|---|---|---|---|---|---|---|---|
| (min) | Mean | RSD % | Mean | RSD % | Mean | RSD % | Mean | RSD % |
| 5 | 72 | 8.2 | 73 | 5.3 | 74 | 5.2 | 73 | 6.3 |
| 10 | 85 | 6.0 | 87 | 4.0 | 88 | 2.6 | 87 | 4.4 |
| 15 | 91 | 5.0 | 92 | 3.6 | 93 | 2.3 | 92 | 3.8 |
| 20 | 93 | 4.9 | 95 | 3.7 | 96 | 2.8 | 95 | 3.9 |
| 30 | 96 | 4.7 | 98 | 3.7 | 98 | 2.6 | 97 | 3.8 |

TABLE 8

| Time | Lot A (n = 12) | | Lot B (n = 12) | | Lot C (n = 12) | | Pooled (n = 36) | |
|---|---|---|---|---|---|---|---|---|
| (min) | Mean | RSD % | Mean | RSD % | Mean | RSD % | Mean | RSD % |
| 5 | 68 | 4.9 | 72 | 3.5 | 62 | 4.0 | 67 | 7.5 |
| 10 | 80 | 3.4 | 89 | 1.1 | 74 | 2.2 | 81 | 8.1 |
| 15 | 85 | 2.7 | 93 | 0.7 | 80 | 1.7 | 86 | 6.7 |
| 20 | 88 | 2.5 | 95 | 0.6 | 84 | 1.7 | 89 | 5.2 |
| 30 | 92 | 3.1 | 95 | 0.5 | 89 | 2.1 | 92 | 3.6 |

The results of this example demonstrate that the aprepitant emulsion prepared according to the method of the invention contains less variation in aprepitant dissolution as compared to the commercially available CINVANTI™ (aprepitant) emulsion.

### EXAMPLE 4

This example demonstrates the globule size distribution of an emulsion of the invention as compared to the commercially available CINVANTI™ (aprepitant) emulsion.

Three lots of aprepitant emulsion vials prepared as described in Example 1 were placed into stability chambers under refrigerated temperature (5° C. ±3° C./ambient humidity) and stored for 6 months prior to globule size analysis. Three lots of commercially available CINVANTI™ (aprepitant) emulsion that had been stored according to package insert (refrigerated at 2° C.-8° C.) and were aged approximately 13 months (lots A and C) and 16 months (lot B) also were examined according to the following method.

Samples containing approximately 7.2 mg/mL aprepitant were tested for globule size distribution as per requirements of USP <729> method I by laser diffraction using a Malvern Mastersizer 3000. The values for D₅₀ and SPAN were determined, where SPAN represents (D₉₀-D₁₀)/D₅₀ and D₁₀, D₅₀, and D₉₀ represent globule diameters with cumulative 10%, 50%, and 90% of globules in a sample below that size, respectively. The results for D₅₀ and SPAN of 10 samples of each lot of exemplary emulsion and 10 samples of each lot of CINVANTI™ (aprepitant) emulsion are summarized in Table 9.

TABLE 9

| | |
|---|---|
| Example | CINVANTI ^{™} |

| Parameter | Mean | SD (σ) | Mean | SD (σ) |
|---|---|---|---|---|
| D₅₀ | 52.9 | 0.5559 | 52.6 | 0.6888 |
| SPAN | 0.662 | 0.09896 | 0.709 | 0.23341 |

The results of this example demonstrate that the aprepitant emulsion prepared according to the method of the invention contains less variation in globule size distribution as compared to the commercially available CINVANTI™ (aprepitant) emulsion.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing an emulsion, the process comprising combining a dry mixture containing at least one active pharmaceutical ingredient (API) and at least one solid emulsifier with an oil comprising at least one co-emulsifier, and, optionally, an additional quantity of the solid emulsifier, and mixing to produce an oil phase; combining the oil phase with an aqueous phase comprising water, at least one tonicity agent, and at least one pH modifier; and, homogenizing the oil and aqueous phases to produce a pharmaceutically acceptable emulsion, wherein the co-emulsifier is present in the emulsion at a pharmaceutically acceptable concentration without removing excess co-emulsifier following emulsification.

2. The process of claim 1, comprising (a) combining the API with a solid emulsifier to produce a dry mixture comprising the API and solid emulsifier, (b) combining an oil with a co-emulsifier and, optionally an additional quantity of the solid emulsifier, to produce a first oil phase, (c) combining the dry mixture with the first oil phase to produce a second oil phase, (d) combining water, a tonicity agent, and a pH modifier to produce an aqueous phase, and (e) homogenizing the second oil and aqueous phases to produce the emulsion without removing co-emulsifier following emulsification.

3. The process of claim 1, wherein the co-emulsifier comprises a volatile co-emulsifier, and the first oil phase produced in step (b) comprises the volatile co-emulsifier in an overage of no more than about 10% more than the total quantity of volatile co-emulsifier present in the emulsion following emulsification.

4. The process of claim 1, wherein the dry mixture produced in step (a) comprises a portion of total amount of solid emulsifier used in the emulsion, and the remainder of solid emulsifier is present in step (c).

5. (canceled)

6. The process of claim 1, wherein the API has a particle size D₉₀ of less than about 100 μm.

7. The process of claim 1, wherein the emulsion comprises from about 1 wt. % to about 10 wt. % of a volatile co-emulsifier, and the amount of volatile co-emulsifier used in step (b) is no more than about 2% to 8% greater than the total amount of volatile co-emulsifier present in the emulsion following emulsification.

8-9. (canceled)

10. The process of claim 1, wherein step (c) further comprises applying a vacuum to a vessel containing the second oil phase, backfilling the vessel with an inert gas, and heating the vessel until complete dissolution of the dry mixture in the second oil phase.

11-12. (canceled)

13. A process for preparing an emulsion, the process comprising (a) combining aprepitant with an egg yolk lecithin to produce a dry mixture comprising the aprepitant and egg yolk lecithin, (b) combining an oil, ethanol, and, optionally, egg yolk lecithin, to form a first oil phase, (c) combining the dry mixture with the first oil phase to form a second oil phase, (d) combining water, sucrose, and oleic acid or a salt thereof to form an aqueous phase, and (e) homogenizing the second oil and aqueous phases to produce the emulsion without removing ethanol following emulsification

14. (canceled)

15. The process of claim 13, wherein the emulsion comprises from about 1 wt. % to about 10 wt. % ethanol, and the amount of ethanol used in step (b) is no more than about 2% to 8% greater than the total amount of ethanol present in the emulsion following emulsification.

16-19. (canceled)

20. The process of claim 13, wherein the aprepitant has a particle size D₉₀ of less than about 100 μm prior to step (a).

21. The process of claim 13, wherein the aprepitant combined in step (a) comprises a mixture of polymorph Form I and polymorph Form II of aprepitant.

22. The process of claim 13, wherein the aprepitant combined in step (a) comprises no more than about 80 wt. % polymorph Form I of aprepitant and no less than about 20 wt. % polymorph Form II.

23. (canceled)

24. The process of claim 13, wherein the aprepitant combined in step (a) comprises from about 70 wt. % to about 80 wt. % polymorph Form I and from about 20 wt. % to about 30 wt. % polymorph Form II of aprepitant.

25. (canceled)

26. An emulsion prepared by the process according to claim 1.

27. (canceled)

28. The emulsion of claim 26, wherein the API comprises aprepitant, and the emulsion remains free of visible aprepitant crystals following storage at 5° C., or room temperature for at least about 1 year.

29. (canceled)

30. The emulsion of claim 26, wherein the API comprises aprepitant, and the emulsion contains about 0.2% or less of total aprepitant-related impurities following storage of the emulsion under refrigerated temperature and/or room temperature conditions for at least about 6 months.

31. (canceled)

32. The emulsion of claim 26, wherein the API comprises aprepitant, and the emulsion contains about 94% or more of the labeled concentration of aprepitant after storage under refrigerated temperature and/or room temperature conditions for at least about 6 months.

33. The emulsion of claim 26, wherein API comprises aprepitant, and the emulsion contains about 98% or more of the labeled concentration of ethanol after storage under refrigerated temperature and/or room temperature conditions for at least about 6 months.

34. The emulsion of claim 26, wherein the API comprises aprepitant, and the emulsion releases about 70% or more of the aprepitant present in the emulsion within 5 minutes in a USP apparatus IV flow through cell dissolution tester.

35. (canceled)

36. The emulsion of claim 26, wherein the API comprises aprepitant, and the emulsion has a SPAN ([D₉₀-D₁₀]/D₅₀) of about 0.70 or less by laser diffraction analysis.

37. (canceled)
